(19) United States
(12) Patent Application Publication
(10) Pub. No.: US 2021/0106804 A1
(43) Pub. Date: Apr. 15, 2021

(54) KEYED FLUID LINE CONNECTORS AND METHODS

(71) Applicant: Merit Medical Systems, Inc., South Jordan, UT (US)

(72) Inventors: Gregory R. McArthur, Sandy, UT (US); Richard P. Jenkins, Bluffdale, UT (US); Benjamin Cleon Skousen, Saratoga Springs, UT (US); David W. Johnson, South Jordan, UT (US)

(21) Appl. No.: 17/067,301

(22) Filed: Oct. 9, 2020

Related U.S. Application Data
(60) Provisional application No. 62/914,300, filed on Oct. 11, 2019.

(51) Int. Cl. A61M 39/10

(52) U.S. Cl. CPC A61M 39/105; A61M 2207/00; A61M 2205/582; A61M 2205/581

(57) ABSTRACT

Medical connectors, including connectors for fluidly coupling fluid lines are disclosed. Some connectors may be configured for coupling fluid lines together including keying features to enable coupling of connector pairs having corresponding keying features and prevent coupling of connector pairs having non-corresponding keying features.

### RELATED CASES

This application claims priority to U.S. Provisional Application No. 62/914,300, filed on Oct. 11, 2019, and titled “Keyed Fluid Line Connectors and Methods,” which is hereby incorporated by reference in its entirety.

### TECHNICAL FIELD

The present disclosure relates generally to the field of medical devices. More specifically, the present disclosure relates to fluid connector assemblies that can be used to couple components of a medical device or assembly to one another, including systems comprising keying configurations configured to couple connector pairs having corresponding key configurations and prevent coupling of connector pairs having non-corresponding key configurations. Related methods are also disclosed.

### BRIEF DESCRIPTION OF THE DRAWINGS

FIG. 1 is a perspective view of a connector pair of a system of keyed fluid line connectors including an “A” connector and a “B” connector according to a first embodiment.

FIG. 2 is a perspective view of a portion of the system of keyed fluid line connectors of FIG. 1 showing an exploded assembly view of the “B” connector.

FIG. 3A is a perspective view of the connector pair of FIG. 1 showing the “A” connector positioned in coupling alignment with the “B” connector.

FIG. 3B is a perspective view of the connector pair of FIG. 1 showing the “A” connector partially disposed within an aperture of the “B” connector.

FIG. 3C is a perspective view of the connector pair of FIG. 1 showing of the system of keyed fluid line connectors of FIG. 1 showing the “A” connector fully inserted within an aperture of the “B” connector but not rotated relative to the “B” connector.

FIG. 3D is a perspective view of the connector pair of FIG. 1 showing the “A” connector fully inserted and rotated relative to the “B” connector.

FIG. 4A is a perspective section view of the connector pair in the configuration shown in FIG. 3B, cut along sectioning lines 4A-4A of FIG. 3B.

FIG. 4B is a perspective section view of the connector pair in the configuration of FIG. 3C cut along sectioning lines 4B-4B of FIG. 3C.

FIG. 4C is a perspective section view of the connector pair in the configuration of FIG. 3D cut along sectioning lines 4C-4C of FIG. 3D.

FIG. 5 is a perspective view of a system of keyed line fluid connectors comprising a plurality of the connector pairs of FIG. 1 further including a manifold coupled to the plurality of “B” connectors.

FIG. 6 is a perspective view of another embodiment of a connector pair of a system of keyed fluid line connectors including an “A” connector and a “B” connector.

FIG. 7 is an exploded perspective view of an insert of a plastic injection mold base.

FIG. 8 is a top front perspective view of a connector pair of a system of keyed fluid line connectors including an “A” connector and a “B” connector, according to one embodiment of the present disclosure.

FIG. 9 is a top rear perspective view of the system of FIG. 8.

FIG. 10 is a perspective view of the system of keyed fluid line connectors of FIG. 8 in a coupled configuration.

FIG. 11 is a perspective view of the “A” connector of FIG. 8.

FIG. 12 is a manifold of the system of keyed fluid line connectors, according to an embodiment of the present disclosure.

FIG. 13 illustrates a plurality of keyed fluid line connectors coupled to the manifold of FIG. 12.

### DETAILED DESCRIPTION

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure but is merely representative of various embodiments. While the various aspects of the embodiments are presented in drawings, the drawings are not necessarily drawn to scale unless specifically indicated.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use or closest to a manifold. The proximal end refers to the opposite end, or the end nearest the practitioner during use or farthest from the manifold.

“Fluid” is used in its broadest sense, to refer to any fluid, including both liquids and gases as well as solutions, compounds, suspensions, etc., which generally behave as fluids.

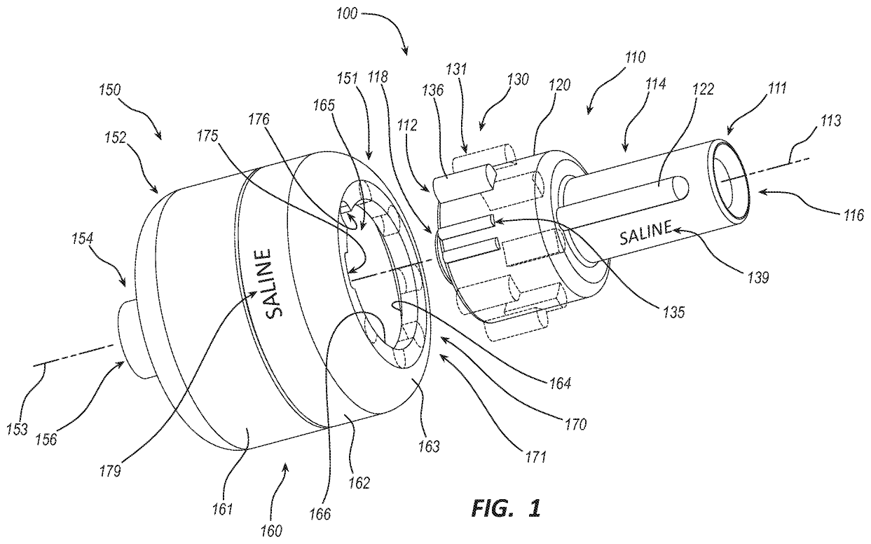

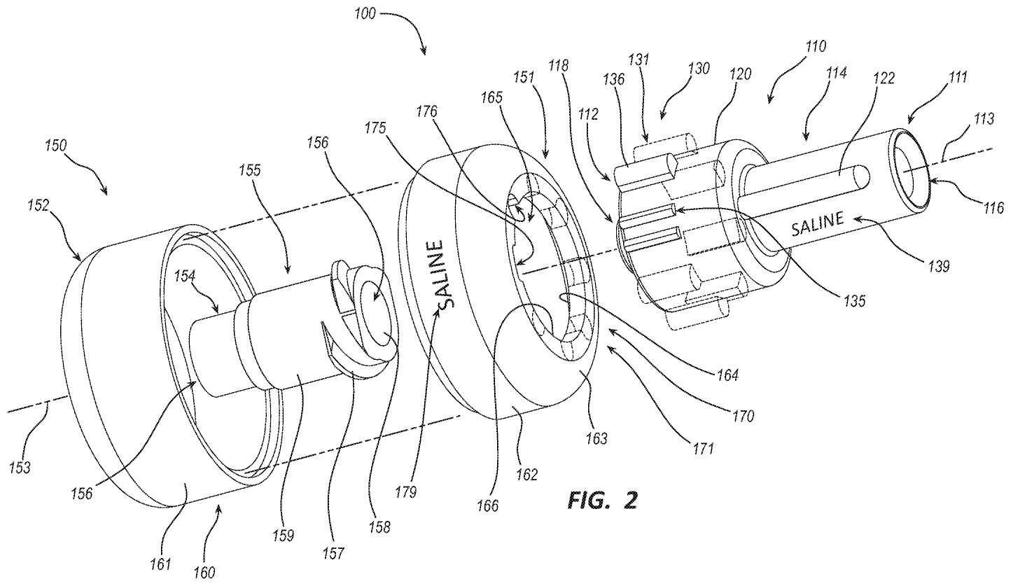

FIGS. 1 and 2 illustrate a system of keyed fluid line connectors 100 according to a first embodiment. FIG. 1 is a perspective view of the system of keyed fluid line connectors 100 illustrating one of a plurality of “A” connectors 110 and one of a plurality of “B” connectors 150. When describing a mating set of connectors, connectors 110 and 150 may each be referred to either of a “first connector” or “second connector” and/or “key connector” or “key receiving connector.” FIG. 2 is a perspective view of the system of keyed fluid line connectors 100 according to FIG. 1 further illustrating the “B” connector in an exploded assembly view. Throughout this disclosure, the “A” connector 110 and the “B” connector 150 may be referred to in the singular. However, it is to be understood that disclosure given in connection with an “A” connector 110 may refer or analogously apply to each of a plurality of “A” connectors and disclosure given in connection with a “B” connector 150 may refer or analogously apply to each of a plurality of “B” connectors 150. The “A” connectors 110 and the “B” connectors 150 are configured to couple together and may comprise corresponding mating features to couple the connectors 110 and 150 together. While the disclosure below refers to the mating features being on a specific connector, these mating features may be disposed on either the “A” connectors 110 or the “B” connectors 150.

The “A” connector 110 comprises a proximal end 111, a distal end 112, and a longitudinal axis 113. Similarly, the “B” connector 150 comprises a proximal end 151, a distal end 152, and a longitudinal axis 153. The “A” connector 110 comprises a fluid lumen 116 disposed along the longitudinal axis 113 and the “B” connector 150 comprises a fluid lumen 156 disposed along the longitudinal axis 153. The “A” connector 110 and “B” connector 150 may be configured to couple to each other when the longitudinal axes 113, 153 are collinearly disposed.

The “A” connector 110 may comprise coupling threads 117 disposed about the longitudinal axis 113, and the “B” connector 150 may comprise complimentary coupling threads 157 disposed about the longitudinal axis 153. Engagement of the coupling threads 117, 157 via rotation of the “A” connector with respect to the “B” connector may facilitate coupling of the “A” connector 110 with the “B” connector 150 and may inhibit inadvertent decoupling. In the illustrated embodiment, the coupling threads 117 may be internal threads, and the coupling threads 157 may be external threads. In other embodiments, the coupling threads 117 may be external threads, and the coupling threads 157 may be internal threads.

The “A” connector 110 may comprise a tapered portion 118 disposed about the longitudinal axis 113, and the “B” connector 150 may comprise a complimentary or mating tapered portion 158 disposed about the longitudinal axis 153. The tapered portions 118, 158 may be configured to form a fluid tight seal between the fluid lumen 116 of the “A” connector 110 and the fluid lumen 156 of the “B” connector 150 when the tapered portions 118, 158 are advanced toward each other or seated against each other. In the illustrated embodiment, the tapered portion 118 may be a male taper and the tapered portion 158 may be a female taper. In other embodiments, the tapered portion 118 may be female and the tapered portion 158 may be male. In some embodiments, the “A” connector 110 and the “B” connector 150 may be complimentary fluid connectors including fittings comprising Luer tapers and connectors complying with at least portions of the ISO 80369-7 standard.

The “A” connector 110 comprises a proximal connecting region 114 configured to facilitate coupling of the “A” connector 110 to medical devices or components, such as tubing segments, syringes, etc., for example, so as to enable the “A” connector 110 to be in fluid communication with a medical fluid source, for example. The “B” connector 150 also comprises a distal connecting region 154 configured to facilitate coupling of the “B” connector 110 to medical devices or components so as to enable the “B” connector 110 to be in fluid communication with a desired destination such as a patient or a medical fluid container, for example.

In some instances, the system of keyed fluid line connectors 100 may be used in conjunction with a plurality of medical fluids. For example, a patient therapy or medical procedure may comprise the transfer of two or more medical fluids. In such instances, fluid lines configured to deliver the two or more medical fluids may be coupled to the patient or other destination at the same time or at varied times. It may also be the case that the two or more medical fluids are delivered via automatically controlled pumps and/or valves. In such instances, a plurality of connector pairs may be used in completing a plurality of fluid circuits between multiple medical fluid sources and destinations. Where the procedure constitutes multiple connector pairs, a misconnection between connector pairs may occur. As such, mechanisms to prevent misconnections may be advantageous. The system of keyed fluid line connectors 100 as illustrated and described below may provide such an advantage.

The “A” connector 110 and the “B” connector 150 of the system of keyed fluid line connectors 100 may comprise common complimentary portions such as the coupling threads 117, 157 and the tapered portions 118, 158 across the pluralities of “A” connectors 110 and “B” connectors 150. In addition to the common complimentary portions, the system of keyed fluid line connectors 100 may also comprise a plurality of key configurations 130 and a plurality of key receiving configurations 170. The plurality of key configurations 130 may be unique with respect to each other and the plurality of key receiving configurations 170 may be unique with respect to each other. The plurality of key configurations 130 may correspond to the plurality of key receiving configurations 170 in a one to one relationship such that each key configuration 130 may only be received in one key receiving configuration 170 and each key receiving configuration 170 may only receive one key configuration 130. In some embodiments, the plurality of key configurations 130 may correspond to the plurality of key receiving configurations 170 in a two or more to one relationship such that more than one key configuration 130 may be received in one key receiving configuration 170. Similarly, in other embodiments, the plurality of key configurations 130 may correspond to the plurality of key receiving configurations 170 in a one to more than one relationship such that one key configuration 130 may be received in more than one key receiving configuration 170.

In the illustrated embodiment, each “A” connector 110 may comprise one of the plurality of key configurations 130, and each “B” connector may comprise one of the plurality of key receiving configurations 170. As such, each “A” connector 110 with its key configuration 130 can couple to each “B” connector 150 having the corresponding key receiving configuration 170 and cannot couple to each “B” connector 150 having a non-corresponding key receiving configuration 170. Similarly, each “B” connector 150 with its key receiving configuration 170 can couple to each “A” connector 110 having the corresponding key configuration 130 and cannot couple to each “A” connector 110 having a non-corresponding key configuration 130.

Each “A” connector 110 may comprise a collar 120 having an external shape 131 defining the key configuration 130, and each “B” connector 150 may comprise an aperture 165 having an internal shape 171 defining the key receiving configuration 170. As such, an “A” connector 110 may couple to a “B” connector 150 only if the collar 120 is receivable (may be disposed) within the aperture 165. In other words, the “A” connector 110 may couple to the “B” connector 150 only if the external shape 131 of the collar 120 corresponds to, or fits within, the internal shape 171 of the aperture 165. The external shape 131 and the corresponding internal shape 171 may be square, triangular, hexagonal, oval, round, rectangular, or any other suitable shape that may or may not be symmetrical.

In the illustrated embodiment, the external shape 131 of the collar 120 may be cylindrical having at least a first protrusion 135 and a second protrusion 136 disposed on and extending away from the cylindrical surface. The first protrusion 135 and the second protrusion 136 may be disposed at different angular locations around the collar 120 so as to define distinctly different (unique) external shapes 131 of the collar 120. As such, the distinctly different external shapes 131 of the collar 120 may define the plurality of key configurations 130. For example, as shown FIG. 1, the second protrusion 136 is positioned adjacent the first protrusion 135 defining one key configuration 130. Additional key configurations 130 may be defined by additional angular positions of the second protrusion 136 relative to the first protrusion 135 as illustrated in FIG. 1 with phantom lines. The number of key configurations 130 as defined by the distinct angular positions of the second protrusion 136 relative to the first protrusion 135 may be 2, 3, 4, 5, 6, or more.

The first protrusion 135 and the second protrusion 136 may have the same or different shapes. In the illustrated embodiment, the second protrusion 136 is shown as a rib having a height extending radially away from the collar 120, a width extending around the collar 120, and a length extending proximally away from a distal edge of the collar 120. The first protrusion 135 is shown as a pair of ribs having a height shorter than the height of the second protrusion 136, a width (defined by the spacing of the ribs) wider than the second protrusion 136, and a length which may be equal to the length of the second protrusion 136. Embodiments having protrusions of other relative heights, widths, and lengths as well as other shapes and longitudinal positions of the first protrusion 135 and the second protrusion 136 are included within this disclosure. Embodiments having a single protrusion, or more than two protrusions are also included in this disclosure.

In the illustrated embodiment, the internal shape 171 of the aperture 165 of the “B” connector may comprise an internal perimeter 166 that is circular so as to correspond to the cylindrical shape of the collar 120. The collar 120 and the aperture 165 may be sized to provide for a sliding fit between the collar 120 and the aperture 165. The internal perimeter 166 may comprise a first notch 175 and a second notch 176 to correspond to the first protrusion 135 and the second protrusion 136. In an analogous fashion to the “A” connector 110, the first notch 175 and the second notch 176 may be disposed at different angular locations around the internal perimeter 166 of the aperture 165 so as to define distinctly different (unique) internal shapes 171 of the aperture 165. As such, the distinctly different internal shapes 171 of the aperture 165 may define the plurality of key receiving configurations 170. The shapes and sizes of the first notch 175 and the second notch 176 may also correspond to (closely match) the shapes and sizes of the first protrusion 135 and the second protrusion 136.

The “B” connector 150 may comprise a connecting portion 155 and a shell 160 as illustrated in FIG. 2. The shell 160 may be formed of a distal shell portion 161 and a proximal shell portion 162. The shell 160, or more specifically the distal shell portion 161, is coupled to the hub 159 of the “B” connector 150. The proximal shell portion 162 is coupled to the distal shell portion 161 and extends proximally beyond the proximal end of the hub 159 of the connecting portion 155. The proximal shell portion 162 comprises a proximal wall 163. The aperture 165 is disposed though the proximal wall 163 to the internal surface 164. The shell 160 is sized and shaped to provide for disposition and rotation of the collar 120 within the shell 160. The shell 160 may substantially enclose a proximal end of the fluid lumen 156 and the tapered portion 158 and thus protect the proximal end of the fluid lumen 156 and the tapered portion 158 from inadvertent contact which may result in contamination of the fluid lumen 156.

The outer shape of the shell 160 may be cylindrical, polygonal, or some other suitable shape. The shape of the shell 160 may also be non-symmetrical or comprise other features such as a textured surface, ribs, bumps, etc. The shape of the shell 160 may facilitate orientation determination of the “B” connector 150 by the practitioner via touch with the thumb and/or fingers. The shape of the shell 160 may also facilitate the application of a torque by the practitioner. In some embodiments, the external shape of the shell 160 may also be associated with the key receiving configuration 170. The plurality of key receiving configurations 170 may correspond to a plurality of external shapes of the shell 160. Similarly, an external shape of the shell 160 may align with, or otherwise indicate, the angular orientation of the key receiving configuration 170.

In some embodiments, the coupling interface between the distal shell portion 161 and the hub 159 may comprise an angular alignment feature, for example a protrusion within a notch (not shown), to facilitate consistent angular alignment of the distal shell portion 161 relative to the connecting portion 155 during assembly. Similarly, the coupling interface between the proximal shell portion 162 and distal shell portion 161 may comprise an angular alignment feature, for example a protrusion within a notch (not shown), to facilitate consistent angular alignment of the proximal shell portion 162 relative to the distal shell portion 161. As such, the angular position of the aperture 165 relative to the coupling threads 157 may be consistent across the plurality of “B” connectors 150. Angular alignment between the aperture 165 and the coupling threads 157 may provide for consistent angular alignment of the coupling threads 117, 157 of the “A” connector 110 and the “B” connector 150, respectively, upon insertion of the collar 120 within the aperture 165.

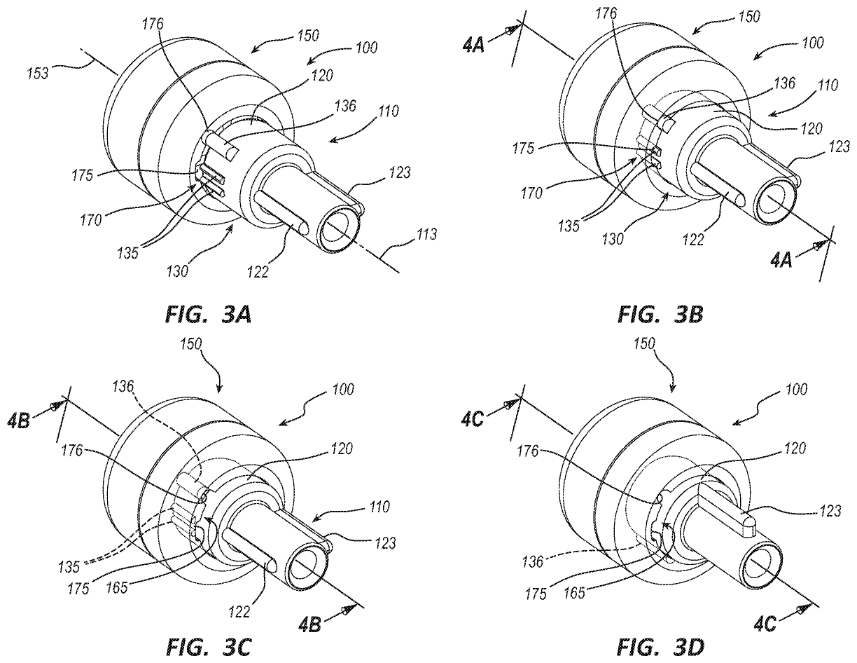

The “A” connector 110 may comprise a first wing 122 disposed proximal of the collar 120 which may facilitate the application of torque by a practitioner during coupling of the “A” connector 110 with the “B” connector 150. The first wing 122 may be disposed in alignment with longitudinal axis 113 and may extend away from the longitudinal axis 113. The first wing 122 may be disposed in alignment with the first protrusion 135 or the second protrusion 136 and or may be disposed consistently in an angularly offset position from the first protrusion 135 or the second protrusion 136. The “A” connector 110 may also comprise a second wing 123 (see FIG. 3A). The second wing 123 may be disposed opposite the first wing 122. The first wing 122 and the second wing 123 may have different shapes allowing a practitioner to distinguish the first wing 122 from the second wing 123 by touch with the thumb and/or fingers, and thereby assess the orientation of the “A” connector 110 by touch. As such, the practitioner may not need to assess the orientation of the “A” connector 110 visually. The first wing 122 or the second wing 123 may extend radially farther from the longitudinal axis 113 than the other or may extend proximally farther from the distal end of the collar 120 than the other. Other differences in shape and size may also facilitate orientation determination via touch such as texture, width, or any other suitable difference. Relative angular positioning of the first wing 122 relative to the second wing 123 may also facilitate orientation determination via touch. In other words, the practitioner may be able align the key configuration 130 of the “A” connector 110 with the key receiving configuration 170 of the “B” connector 150 without visual assessment.

In some embodiments, the “A” connector 110 and/or the “B” connector 150 may comprise one or more indicia 139, 179 respectively. The one or more indicia 139, 179 may indicate if the key configuration 130 of the “A” connector 110 corresponds to the key receiving configuration 170 of the “B” connector 150. The one or more indicia 139, 179 may also indicate a medical fluid to be disposed through the “A” connector 110 and the “B” connector 150, respectively. Still further, the one or more indicia 139, 179 may indicate the angular orientation of the key configuration 130 of the “A” connector 110 with respect to the key receiving configuration 170 of the “B” connector 150. In some embodiments, a color of the “A” connector 110 and of the “B” connector 150 or a portion of either, may indicate if the key configuration 130 of the “A” connector 110 corresponds to the key receiving configuration 170 of the “B” connector 150. In other words, the plurality of “A” connectors 110 may comprise a plurality of colors with each key configuration 130 having a different color. In other embodiments, the plurality “B” connectors 150 may comprise a plurality of colors with each key receiving configuration 170 having a different color and the color of each key configuration 130 may be the same as the color of each corresponding key receiving configuration 170. Still in other embodiments, the key configurations 130 and the key receiving configurations 170 may comprise tactile features for identifying pairs of corresponding key configurations 130 and key receiving configurations 170 and/or indicating the angular orientation of the key configuration 130 or key receiving configuration 170.

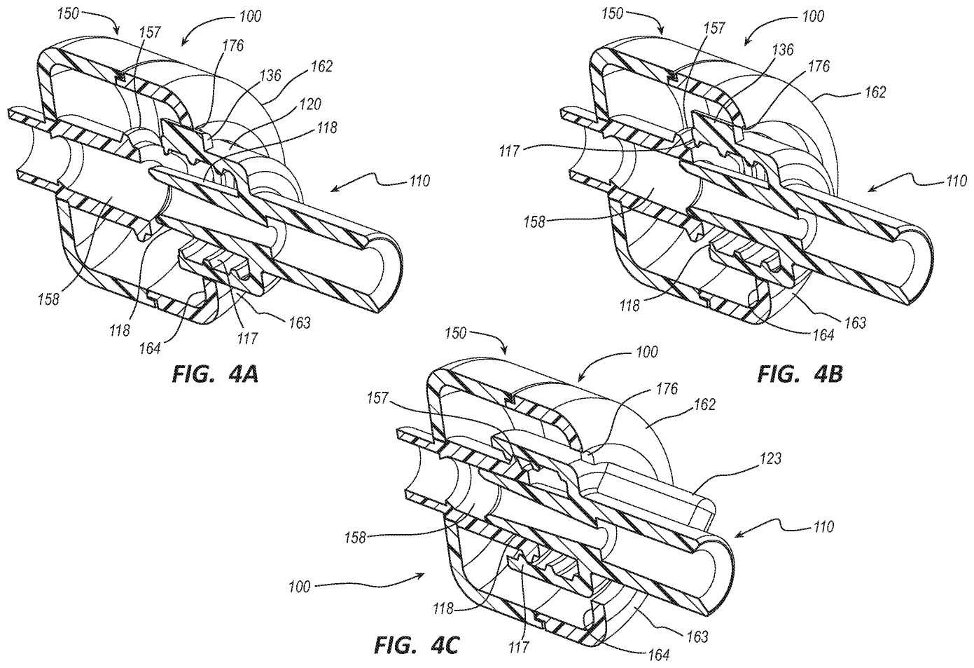

FIGS. 3A-3D depict the “A” connector 110 with its key configuration 130 and the “B” connector 150 with its corresponding key receiving configuration 170 in progressing states of coupling. FIGS. 4A-4C are section views of FIGS. 3B-3D. Referring to FIG. 3A, the “A” connector 110 is depicted in alignment with the “B” connector such that the longitudinal axis 113 of the “A” connector 110 is collinear with the longitudinal axis 153 of the “B” connector 150. The “A” connector 110 is also in angular alignment with the “B” connector such that the key configuration 130 is angularly aligned with the key receiving configuration 170, or more specifically, the first protrusion 135 is aligned with the first notch 175, and the second protrusion 136 is aligned with the second notch 176.

FIGS. 3B and 4A illustrate the “A” connector 110 partially coupled to the “B” connector such that the collar 120 of the “A” connector is partially disposed within the aperture 165 of the “B” connector 150. FIG. 4A shows distal portions of the collar 120 and the second protrusion 136 disposed beyond the internal surface 164 of the proximal wall 163. The coupling threads 117 of the “A” connector 110 are shown longitudinally spaced away from the coupling threads 157 of the “B” connector 150 and the external tapered portion 118 is shown longitudinally spaced away from the internal tapered portion 158. As the first protrusion 135 (not shown in FIG. 4A) and the second protrusion 136 are disposed within the first notch 175 (also not shown in FIG. 4A) and the second notch 176, respectively, rotation of the “A” connector 110 relative to the “B” connector 150 is prevented.

FIGS. 3C and 4B illustrate the “A” connector 110 further displaced toward the “B” connector to the point where the coupling threads 117 contact the coupling threads 157 preventing further direct insertion. At this illustrated state of coupling, the collar 120 of the “A” connector is further disposed within the aperture 165 of the “B” connector 150 such that the first protrusion 135 (not shown in FIG. 4B) and the second protrusion 136 are completely disposed beyond the internal surface 164 of the proximal wall 163. The coupling threads 117 of the “A” connector 110 are shown in contact with the coupling threads 157 of the “B” connector 150 and the external tapered portion 118 is shown partially disposed within the internal tapered portion 158. As the first protrusion 135 and the second protrusion 136 are distally disposed beyond the internal surface 164, the first protrusion 135 and the second protrusion 136 are disposed distally beyond, or out of, the first notch 175 and the second notch 176. As such, rotation of the “A” connector 110 relative to the “B” connector 150 is allowed.

In some embodiments, rotation of the “A” connector 110 relative to the “B” connector 150 while the first protrusion 135 and the second protrusion 136 are distally disposed beyond the internal surface 164, may not coincide with engagement of the coupling threads 117, 157. As such, rotation of the “A” connector 110 relative to the “B” connector 150 may be permitted in both the clockwise and counter-clockwise directions up to or beyond 360 degrees. In other embodiments, rotation of the “A” connector 110 relative to the “B” connector 150 while the first protrusion 135 and the second protrusion 136 are distally disposed beyond the internal surface 164, may only be permitted in the clockwise direction in conjunction with engagement of the coupling threads 117, 157.

In some embodiments, the system of keyed fluid line connector 100 may be configured so that at the state of coupling illustrated in FIGS. 3C and 4B, the coupling threads 117, 157 are angularly disposed adjacent an initial point of thread engagement. In other words, the angular alignment of the key configuration 130 with the key receiving configuration 170 may also define angular alignment of the coupling threads 117, 157 such that initial engagement of the coupling threads 117, 157 occurs immediately upon clockwise rotation of the “A” connector 110 relative to the “B” connector 150.

FIGS. 3D and 4C illustrate the “A” connector 110 and the “B” connector 150 in a completed stage of coupling. As shown, the coupling threads 117, 157 are engaged and the external tapered portion 118 is sealably coupled to the internal tapered portion 158. The first protrusion 135 (not shown in FIG. 4C) and the second protrusion 136 are further distally disposed beyond the internal surface 164 and are rotated away from the first notch 175 (not shown in FIG. 4C) and the second notch 176.

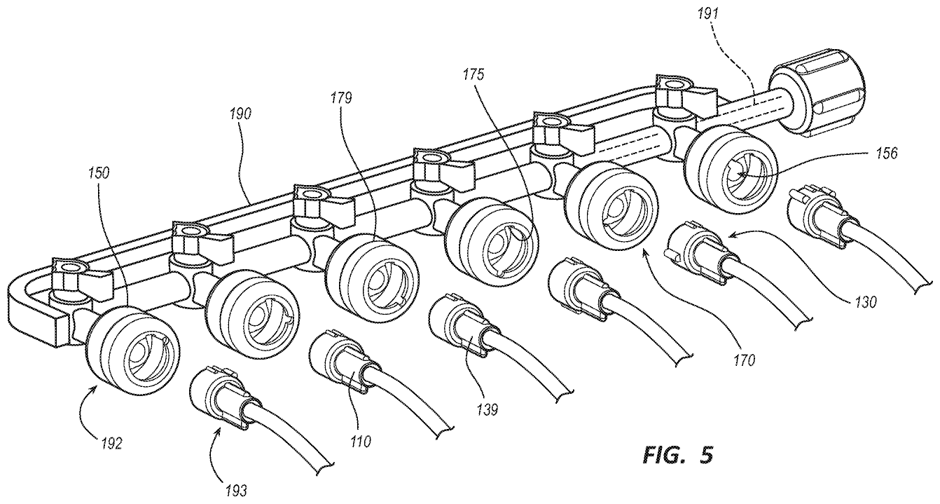

FIG. 5 illustrates the system of keyed fluid line connectors 100 as described above further comprising a manifold 190. In the illustrated embodiment, a subset 192 of six “B” connectors 150 may be coupled to the manifold 190 such that the fluid lumen 156 of each “B” connector 150 may be disposed in fluid communication with a channel or main lumen 191 of the manifold 190. In the illustrated embodiment, the subset 192 of “B” connectors 150 may comprise a subset of key receiving configurations 170 such that each key receiving configuration 170 is unique with respect to the other key receiving configurations 170 across the subset 192. As such, the subset 192 of “B” connectors 150 may only couple to a subset 193 of “A” connectors 110 having corresponding key configurations 130. In other embodiments, two or more of the key receiving configurations across the subset 192 may be the same. Embodiments comprising 2, 3, 4, 5, or more than 6 “B” connectors 150 coupled to the manifold 190 are also within the scope of this disclosure.

The subset 192 of “B” connectors 150 may be coupled to the manifold 190 in a consistent orientation. For example, the “B” connectors 150 may be oriented such that the first notch 175 of each “B” connector is disposed at the top as illustrated in FIG. 5. Other consistent orientations of the “B” connectors 150 are also within the scope of this disclosure. A consistent orientation of the “B” connectors 150 may ease coupling of the “A” connectors by facilitating consistent orientation of each “A” connector 110 upon coupling.

In some embodiments, the subset 192 of “B” connectors 150 and the subset 193 of “A” connectors 110 may include indicia 179, 139, respectively. The indicia may be human or machine readable. The content of the indicia may comprise information related to a patient therapy or medical procedure such as the medical fluids to be transferred, for example. An “A” connector 110 with its key configuration and a “B” connector 150 with a corresponding key receiving configuration may have the same or related indicia content. As such, the indicia may aid in identifying coupleable connector pairs.

In some embodiments, the subset 192 of “B” connectors 150 and the subset 193 of “A” connectors 110 may include colored portions comprising two or more different colors. The colors may be associated with the medical fluids to be transferred in the course of a patient therapy or medical procedure. An “A” connector 110 with its key configuration and a “B” connector 150 with a corresponding key receiving configuration may have the same color. As such, the color may aid in identifying coupleable connector pairs. The colors may also correspond to a color standard of the clinical setting as may be used to identify fluid types such as saline or other medical fluids.

In some embodiments, the manifold 190 having the subset 192 of “B” connectors 150 coupled thereto and the subset 193 of “A” connectors 110 may be packaged together to form a kit. The kit may be configured for a specific patient therapy or medical procedure. The subset 193 of “A” connectors 110 and the subset 192 of “B” connectors 150 may comprise indicia associated with the patient therapy or medical procedure.

Use of the system of keyed fluid line connectors described above may include any of the following steps or processes, each of which may be optional or interchanged. The following steps or processes may be performed in any order unless specifically defined.

Use of the system of keyed fluid line connectors may comprise providing a subset of “A” connectors chosen from a plurality of “A” connectors to couple to a provided subset of “B” connectors chosen from a plurality of “B” connectors for the purpose of, or otherwise to facilitate, the performance of a medical procedure. Each “B” connector of the subset of “B” connectors may comprise a key receiving configuration unique to the key receiving configurations of the other “B” connectors within its subset. Each “A” connector of the subset of “A” connectors may comprise a key configuration corresponding to one of the key receiving configurations of the “B” connectors. In some embodiments, the quantity of “A” connectors within its subset may be equal to the quantity of “B” connectors within its subset. The key configuration of each “A” connector may be unique with respect to the key configurations of the other “A” connectors within its subset, and the key configuration of each “A” connector may corresponded to the key receiving configuration of each “B” connector. In other embodiments, the quantity of “A” connectors within its subset may be greater than the quantity of “B” connectors within its subset, and two or more “A” connectors may have the same key configuration. As such, during a medical procedure, one “A” connector may be disconnected from a “B” connector and replaced with a different “A” connector having the same key configuration.

Use of the system of keyed fluid line connectors may further comprise associating an “A” connector having its key configuration with a “B” connector having the corresponding key receiving configuration. In some embodiments, identifying a key configuration or a key receiving configuration may be difficult without close visual inspection. As such, the associating step may comprise matching a color of at least a portion of the “A” connector with the color of at least a portion of the “B” connector. The associating step may also or alternatively comprise matching indicia content of the “A” connector with corresponding indicia content of the “B” connector.

Use of the system of keyed fluid line connectors may further comprise aligning the “A” connector with the “B” connector. Aligning may comprise angularly orienting the “A” connector about its longitudinal axis such that the key configuration of the “A” connector is angularly aligned with the key receiving configuration of the “B” connector. Aligning may also comprise determining the angular orientation of the “A” connector by feeling the position of one or more wings of the “A” connector when grasped between the thumb and a finger, and adjusting the orientation so that the one or more wings are disposed in a predetermined direction, for example, directed vertically upward. Aligning may further comprise disposing the longitudinal axis of the “A” connector in linear alignment with the longitudinal axis of the “B” connector.

Use of the system of keyed fluid line connectors may further comprise inserting at least a distal portion of a first “A” connector within an aperture disposed at a proximal end of a first “B” connector. Inserting may further comprise disposing one or more protrusions of the first “A” connector or the first “B” connector within one or more notches of the first “A” connector or the first “B” connector wherein when the one or more protrusions of the first “A” connector or the first “B” connector are disposed within the one or more notches of the first “A” connector or the first “B” connector, rotation of the first “A” connector with respect to the first “B” connector is prevented. Inserting may still further comprise distally displacing the one or more protrusions of the first “A” connector or the first “B” connector beyond the one or more notches of the first “A” connector or the first “B” connector such that rotation of the first “A” connector with respect to the first “B” connector is allowed. Inserting may also further comprise disposing coupling threads of the first “A” connector in contact with coupling threads of the first “B” connector such that engagement of the coupling threads occurs immediately upon clockwise rotation of the first “A” connector with respect to the first “B” connector.

Use of the system of keyed fluid line connectors may further comprise rotating the first “A” connector with respect to the first “B” connector wherein, upon rotation of the first “A” connector with respect to the first “B” connector, complimentary coupling threads of the first “A” connector and the first “B” connector draw the first “A” connector toward the first “B” connector which in turn disposes a tapered portion of the first “A” connector into contact with a tapered portion of the first “B” connector establishing a fluid tight seal between the first “A” connector and the first “B” connector.

The method of use may further comprise the coupling of a second “A” connector with a second “B” connector according to the steps described in relation to the first “A” connector and the first “B” connector. The method may further comprise the coupling of three, four, five, six, or more pairs of “A” connectors and “B” connectors according to the steps described in relation to the first “A” connector and the first “B” connector.

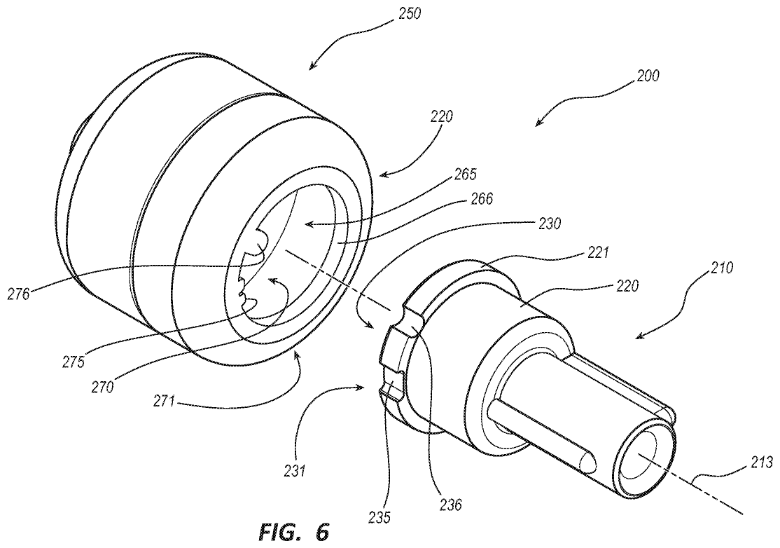

FIG. 6 illustrates another embodiment of a system of keyed fluid line connectors 200 that can, in certain respects, resemble components of the system of keyed fluid line connectors 100 described in connection with FIGS. 1, 2, 3A-3D, 4A-4C, and 5. It will be appreciated that the illustrated embodiments may have analogous features. Accordingly, like features are designated with like reference numerals, with the leading digits incremented to “2.” For instance, the “A” connector is designated as “110” in FIGS. 1, 2, 3A-3D, 4A-4C, and 5, and an analogous “A” connector is designated as “210” in FIG. 6. Relevant disclosure set forth above regarding similarly identified features thus may not be repeated hereafter. Moreover, specific features of the system of keyed fluid line connectors 100 and related components shown in FIGS. 1, 2, 3A-3D, 4A-4C, and 5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system of keyed fluid line connectors 200 of FIG. 6. Any suitable combination of the features, and variations of the same, described with respect to the system of keyed fluid line connectors 100 and components illustrated in FIGS. 1, 2, 3A-3D, 4A-4C, and 5 can be employed with the system of keyed fluid line connectors 200 and components of FIG. 6, and vice versa.

FIG. 6 illustrates a system of keyed fluid line connectors 200 comprising a plurality of “A” connectors 210 and a plurality of “B” connectors 250 of which one “A” connector 210 and one “B” connector 250 are shown in FIG. 6. The system of keyed fluid line connectors 200 also comprises a plurality of key configurations 230 (illustrated as a single key configuration) and a plurality of key receiving configurations 270 (illustrated as a single key receiving configuration). The plurality of key configurations 230 may be unique with respect to each other and the plurality of key receiving configurations 270 may be unique with respect to each other. The plurality of key configurations 230 may correspond to the plurality of key receiving configurations 270 in a one to one relationship such that each key configuration 230 may only be received in one key receiving configuration 270 and each key receiving configuration 270 may only receive one key configuration 230.

In the illustrated embodiment, each “A” connector 210 may comprise one of the plurality of key configurations 230, and each “B” connector 250 may comprise one of the plurality of key receiving configurations 270. As such, each “A” connector 210 with its key configuration 230 can couple to each “B” connector 250 having the corresponding key receiving configuration 270 and cannot couple to each “B” connector 250 having a non-corresponding key receiving configuration 270. Similarly, each “B” connector 250 with its key receiving configuration 270 may couple to each “A” connector 210 having the corresponding key configuration 230 and cannot couple to each “A” connector 210 having a non-corresponding key configuration 230.

Each “A” connector 210 may comprise a collar 220 having an annular ridge 221 disposed adjacent a distal end of the collar 220 and extending radially away from the collar 220. An external shape 231 of the annular ridge 221 may define a key configuration 230. Each “B” connector 250 may comprise an aperture 265 having an internal shape 271. As such, the “A” connector 210 can couple to the “B” connector 250 only if the ridge 221 is receivable within the aperture 265. In other words, the “A” connector 210 can couple to the “B” connector 250 only if the external shape 231 of the ridge 221 corresponds to or fits within the internal shape 271 of the aperture 265. The size of the aperture 265 may be such that when the ridge 221 is disposed within the aperture 265, radial clearance between the ridge 221 and the aperture 265 accommodates sliding displacement and prevents rotation of the ridge 221 relative to the aperture 265. The external shape 231 and the corresponding internal shape 271 may be square, triangular, hexagonal or any other suitable shape.

In the illustrated embodiment, the external shape 231 of the ridge 221 is cylindrical with a first notch 235 and a second notch 236 disposed on the cylindrical surface and extending inward toward the longitudinal axis 213. The first notch 235 and the second notch 236 may be disposed at different angular locations around the ridge 221 so as to define distinctly different external shapes 231 of the ridge 221. As such, the distinctly different external shapes 231 may define the plurality of key configurations 230. For example, as shown in FIG. 6, the second notch 236 is positioned adjacent the first notch 235 defining one key configuration 230. Additional unique key configurations 230 may be defined by additional angular positions of the second notch 236 relative to the first notch 235. The number of key configurations 230 as defined by the distinctly different angular positions of the second notch 236 relative to the first notch 235 may be 2, 3, 4, 5, 6, or more.

The first notch 235 and the second notch 236 may have the same or different shapes. In the illustrated embodiment, the second notch 236 is shown as a rib having a depth extending radially inward from the circumference of the ridge 221 and a width extending around the ridge 221. In the illustrated embodiment, the first notch 235 may comprise a depth that is shallower than the depth of the second notch 236 and width that is wider than the second notch 236. Embodiments having notches of other relative depths and widths as well as other shapes of the first notch 235 and the second notch 236 are included within this disclosure. Embodiments having a single notch or more than two notches are also included in this disclosure.

In the illustrated embodiment, the internal shape 271 of the aperture 265 of the “B” connector 250 may comprise an internal perimeter 266 that is circular so as to correspond to the cylindrical circumference of the ridge 221. The internal perimeter 266 may comprise a first protrusion 275 extending inward of the internal perimeter 266 and a second protrusion 276 also extending inward. The first protrusion 275 and the second protrusion 276 may be sized and shaped to correspond to the first notch 235 and the second notch 236. In similar fashion to the “A” connector 210, the first protrusion 275 and the second protrusion 276 may be disposed at different angular locations around the internal perimeter 266 of the aperture 265 so as to define distinctly different internal shapes 271 of the aperture 265. As such, the distinctly different internal shapes 271 of the aperture 265 may define the plurality of key receiving configurations 270.

Manufacturing the system of keyed fluid line connectors described above may include any of the following steps or processes, each of which may be optional or interchanged. The following steps or processes may be performed in any order unless specifically defined. The manufacturing of medical devices may comprise injection molding of one or more components with thermoplastic materials. The injection molding process may include an injection molding machine and a mold specifically configured to form the component. The expense of the mold may be significant and therefore, it may be advantageous to use one mold to produce multiple components. In such instances, the mold may comprise a mold base configured to accommodate an insert and the insert may be specifically configured to form the component. In some cases, a mold base may be configured to accommodate multiple inserts either individually or as sets. Each insert may be configured to form a specific plastic component such as the “A” connector 110, for example.

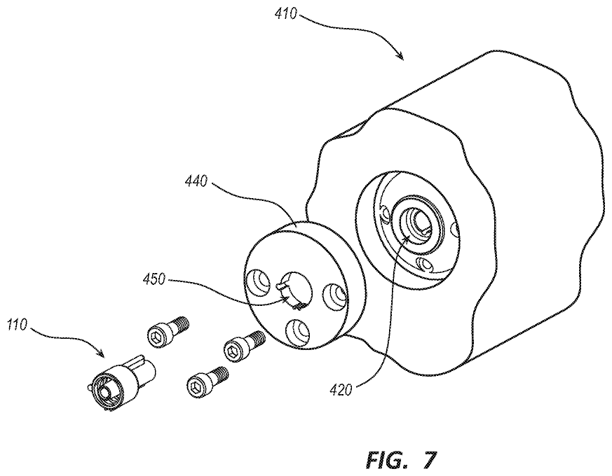

FIG. 7 is an exploded perspective view of a portion of a mold insert 410 having a cavity 420 shaped to form a first exterior portion of the “A” connector 110. Also shown in FIG. 7 is a sub-insert 440 having a cavity 450 shaped to form a second exterior portion of the “A” connector 110. The cavity 420 together with the cavity 450 may therefore form the complete or substantially complete exterior of the “A” connector 110. The first exterior portion as formed by the cavity 420 may comprise portions of the exterior that are common across the plurality of key configurations 130 (see FIG. 1). The second exterior portion as formed by the cavity 450 may comprise portions of the exterior that define the plurality of distinctly different (unique) key configurations 130.

The sub-insert 440 may be one of a plurality of sub-inserts 440 to be installed into the mold insert 410. Each of the plurality of sub-inserts 440 may define and form a different key configuration. In other words, each sub-insert 440 in combination with the mold insert 410 may form the exterior of an “A” connector 110 having one of the plurality of key configurations. As such, the exterior of a plurality of “A” connectors 110 having a plurality of key configurations may be formed by one mold insert 410 in combination with a plurality of sub-inserts 440.

Manufacturing a plurality of “A” connectors 110 having a plurality of unique key configurations 130 may include installing a first sub-insert 440 into the mold insert 410 and molding a first plurality of the “A” connectors 110 having a first key configuration 130. The first sub-insert 440 may then be replaced by a second sub-insert 440. A second plurality of the “A” connectors 110 having a second key configuration 130 may then be molded. The process of replacing the sub-insert 440 and molding a plurality of the “A” connectors 110 may then be repeated so that a plurality of “A” connectors 110 having a plurality of key configurations 130 may be molded.

The process of molding a plurality of proximal shell portions 162 of the “B” connector 150 may comprise a similar process as described above. A replaceable core pin (not shown), as a component of a mold insert 410, may be shaped to form the aperture 165 defining one of the plurality of key receiving configurations 170. As such, a plurality of proximal shell portions 162 having a plurality of key receiving configurations 170 may be molded by molding subsets of the plurality of proximal shell portions 162 wherein each subset is molded with a core pin defining a different key receiving configuration 170.

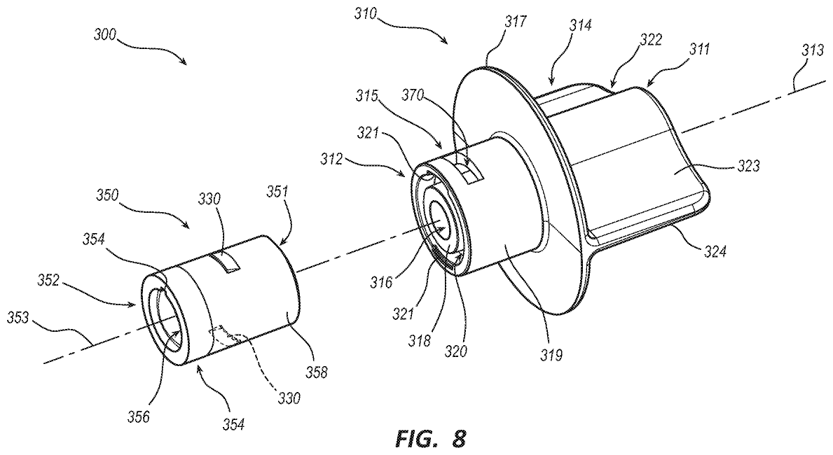

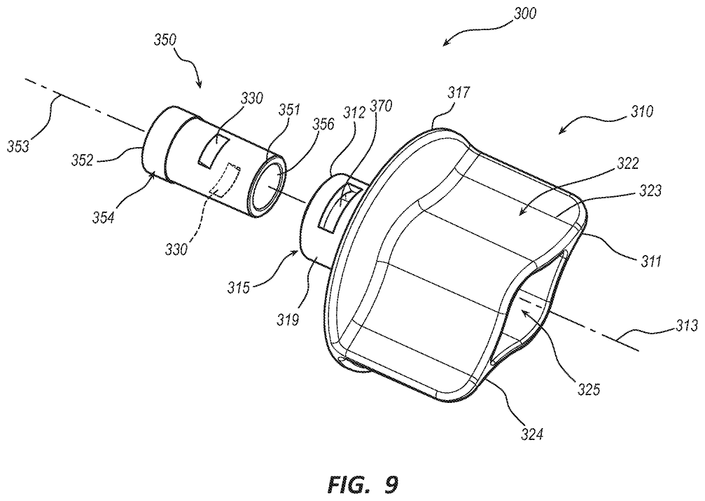

FIGS. 8 and 9 illustrate a system of keyed fluid line connectors 300 according to one embodiment of the present disclosure in an uncoupled configuration. FIG. 8 is a top front perspective view of the system of keyed fluid line connectors 300 illustrating one of a plurality of “A” connectors 310 and one of a plurality of “B” connectors 350. When describing a mating set of connectors, connectors 310 and 350 may each be referred to either of a “first connector” or “second connector” and/or “key connector” or “key receiving connector.” FIG. 9 is a top rear perspective view of the system of keyed fluid line connectors 300 according to FIG. 8. Throughout this disclosure, the “A” connector 310 and the “B” connector 350 may be referred to in the singular. However, it is to be understood that disclosure given in connection with an “A” connector 310 may refer or analogously apply to each of a plurality of “A” connectors and disclosure given in connection with a “B” connector 350 may refer or analogously apply to each of a plurality of “B” connectors 350. The “A” connectors 310 and the “B” connectors 350 are configured to couple together and may comprise corresponding mating features to couple the connectors 310 and 350 together. While the disclosure below refers to the mating features being on a specific connector, these mating features may be disposed on either the “A” connectors 310 or the “B” connectors 350.

The “A” connector 310 comprises a proximal end 311, a distal end 312, and a longitudinal axis 313. Similarly, the “B” connector 350 comprises a proximal end 351, a distal end 352, and a longitudinal axis 353. The “A” connector 310 comprises a fluid lumen 316 disposed along the longitudinal axis 313 and the “B” connector 350 comprises a fluid lumen 356 disposed along the longitudinal axis 353. The “A” connector 310 and “B” connector 350 may be configured to couple to each other when the longitudinal axes 313, 353 are collinearly disposed.

The “A” connector 310 may comprise a proximal portion 314, a distal portion 315, and a flange 317 disposed between the proximal portion 314 and the distal portion 315. The proximal portion 314 comprises a contoured finger grip 322 that enables a user to grip the “A” connector 310 when coupling the “A” connector 310 to the “B” connector 350.

The distal portion 315 comprises a pair of concentric projections that extend distally from the flange 317. The pair of concentric projections include a skirt 319 and a nozzle 318, the nozzle 318 comprising the fluid lumen 316. The skirt 319 is disposed radially outside the nozzle 318 creating a circular groove 320 separating the skirt 319 and the nozzle 318. The skirt 319 has a diameter that is greater than the nozzle 318. The skirt 319 may have a constant radial thickness that extends in the distal direction. The nozzle 318 may be conical and tapered thereby having a varying radial thickness that decreases gradually in the proximal direction.

As discussed above, the “A” connector 310 comprises the nozzle 318 disposed about the longitudinal axis 313, and the “B” connector 350 may comprise a complimentary or mating tapered portion 358 disposed about the longitudinal axis 353. The nozzle 318 and the tapered portion 358 may be configured to form a fluid tight seal between the fluid lumen 316 of the “A” connector 310 and the fluid lumen 356 of the “B” connector 350 when the nozzle 318 and the tapered portion 358 are advanced toward each other or seated against each other. In the illustrated embodiment, the nozzle 318 may be a male taper and the tapered portion 358 may be a female taper. In other embodiments, the nozzle 318 may be female and the tapered portion 358 may be male. In some embodiments, the “A” connector 310 and the “B” connector 350 may be complimentary fluid connectors including fittings comprising Luer tapers and connectors complying with at least portions of the ISO 80369-7 standard.

The “A” connector 310 and the “B” connector 350 of the system of keyed fluid line connectors 300 may comprise common complimentary portions, such as the nozzle 318 and the tapered portion 358 across the pluralities of “A” connectors 310 and “B” connectors 350. Other complimentary portions may include a plurality of key configurations 330 and a plurality of key receiving configurations 370 that couple and secure the “A” connectors 310 to the “B” connectors 350, in other words, mating features. In some embodiments, the plurality of key configuration 330 may be disposed on the “B” connectors 350 and the plurality of key receiving configurations 370 may be disposed on the “A” connectors 310. In some embodiments, the plurality of key configurations 330 may be disposed on the “A” connectors 310 and the plurality of key receiving configurations 370 may be disposed on the “B” connectors 350.

In the illustrated embodiment, the “B” connector 350 may comprise two key configurations 330 that are on opposite sides of the tapered portion 358, with one of the key configurations in phantom lines because it is hidden. The key configurations 330 may be a pair of projections that extend outward of the tapered portion 358 in a radial direction. The key configurations 330 may comprise a wedge shape that extends in a distal direction at an angle away from the tapered portion 358.

The “A” connector 310 may comprises two key receiving configurations 370 disposed on opposite sides of the skirt 319. The key receiving configuration 370 may be windows or apertures for receiving the key configurations 330 of the “B” connectors 350 that extend through the skirt 319 in the radial direction. The window may comprise a shape that corresponds with the shape of the key configuration 330. In other words, if the key configurations 330 are wedge shaped, the window may be rectangular shaped to receive the key configurations 330. The key configurations 330 are configured to snap into the key receiving configurations 370 on the skirt 319. When the key configurations snap into the key receiving configurations 370, the snapping provides an audible and tactile feedback to the user to communicate to the user that the “B” connector 350 has connected to the “A” connector 310.

In some embodiments, the distal end 312 of the “A” connector 310 may comprise a plurality of grooves 321 for directing the key configurations 330 of the “B” connector 350. The grooves 321 may be disposed on a distal end of the skirt 319 and the grooves 321 align in a longitudinal direction with the key receiving configurations 370. Accordingly, the key configurations 330 cannot enter the groove 320 of the “A” connector 310 until the key configurations 330 longitudinally align with the grooves 321 of the skirt 319. The grooves 321 may have an arcuate shape. Thus, the grooves 321 help the user couple the “A” connector 310 to the “B” connector 350 by feel without having to use sight to align the “A” connector 310 and the “B” connector 350. Once the key configurations 330 of the “B” connector 350 align with the grooves 321 of the “A” connector 310, the key configurations 330 may enter the groove 320 and the key configurations 330 may snap into the key receiving configurations 370 of the “A” connector 310.

The “B” connector 350 comprises a distal connecting region 354 configured to facilitate coupling of the “B” connector 350 to medical devices or components so as to enable the “B” connector 350 to be in fluid communication with a desired destination such as a patient or a medical fluid container, for example. The distal connecting region 354 may include a radial groove 355 that is configured to couple to a manifold. In the illustrated embodiment, the radial groove 355 has a rectangular shape. However, the radial groove 355 may be square, triangular, polygonal, and the like.

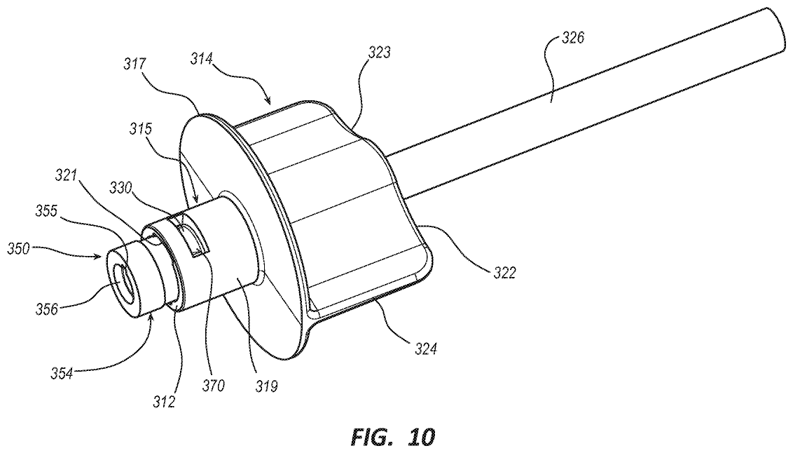

FIG. 10 illustrates the system of keyed fluid line connectors 300 in a coupled configuration. In the coupled configuration, the key configurations 330 of the “B” connector 350 are received into the key receiving configurations 370 of the “A” connectors 310. The proximal end 311 of the “A” connector 310 may be configured to couple to medical devices or components, such as tubing segments, syringes, etc., for example, so as to enable the “A” connector 310 to be in fluid communication with a medical fluid source. In the illustrated embodiment, a tube 326 couples to the proximal end 311 of the “A” connector.” In addition, FIG. 10 illustrates the “A” connector 310 coupled to the tube 326.

As discussed above, the plurality of “A” connectors 310 comprise a number of different sizes and the plurality of “B” connectors 350 also comprises a number of different sizes. The “A” connectors 310 and the “B” connectors 350 are designed to only engage with the corresponding sized connector. The skirt 319 and the nozzle 318 of the “A” connector 310 are sized to only allow connector to a corresponding “B” connector 350. If the “A” connector 310 is too large, the nozzle 318 will not go into the lumen 356 of the “B” connector 350. If the “A” connector is too small, the skirt 319 of the “A” connector will interfere with the outer diameter of the tapered portion 359 of the “B” connector 350, thus preventing connection.

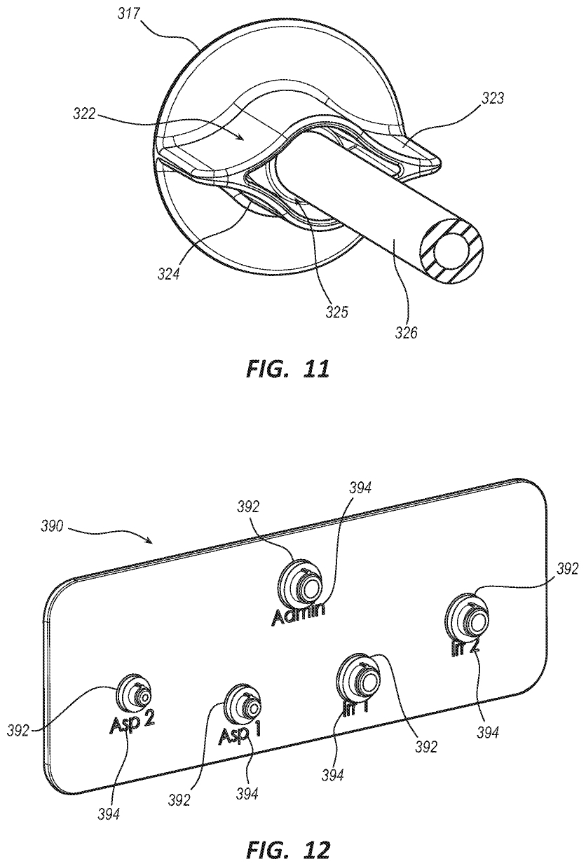

FIG. 11 illustrates the proximal portion 314 of the “A” connector 310. The proximal portion 314 includes the contoured finger grip 322. The contoured finger grip 322 includes a top surface 323 and a bottom surface 324. The contoured finger grip 322 extends from a first lateral side of the “A” connector 310 to a second lateral side of the “A” connector 310 with a bump or hump in the middle. The top surface 323 and a bottom surface 324 define a tubing pocket 325 that is configured to receive the tube 326. The tube 326 may be bonded to the “A” connector 310 and form a seal with the “A” connector 310 to prevent leakage of the fluid disposed within the tube 326. The tubing pocket 325 may be cored out during the molding process.

In some instances, the system of keyed fluid line connectors 300 may be used in conjunction with a plurality of medical fluids. For example, a patient therapy or medical procedure may comprise the transfer of two or more medical fluids. In such instances, fluid lines configured to deliver the two or more medical fluids may be coupled to the patient or other destination at the same time or at varied times. It may also be the case that the two or more medical fluids are delivered via automatically controlled pumps and/or valves. In such instances, a plurality of connector pairs may be used in completing a plurality of fluid circuits between multiple medical fluid sources and destinations. Where the procedure constitutes multiple connector pairs, a misconnection between connector pairs may occur. As such, mechanisms to prevent misconnections may be advantageous in some applications. The system of keyed fluid line connectors 100 as illustrated and described below may provide such an advantage.

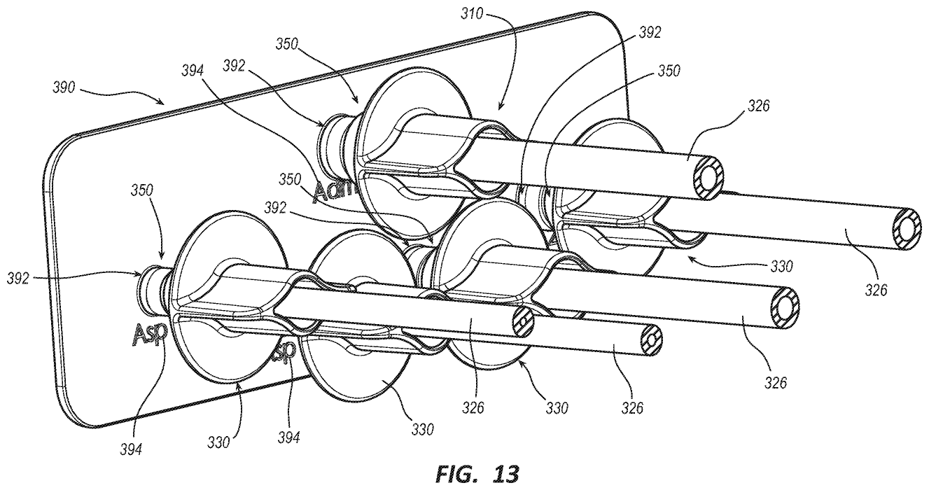

FIG. 12 illustrates the system of keyed fluid line connectors 300 as described above further comprising a manifold 390. In the illustrated embodiment, the manifold 390 comprises a plurality of sockets 392 that are configured to couple to the distal connecting region 354 of the “B” connector 350. Each sockets 392 of the manifold 390 may provide a specific function. For example, the various sockets 392 may provide various types of fluids (saline or other medical fluids), vacuum, etc. Each socket 392 may be sized to receive a different sized “B” connector 350. Accordingly, only the appropriately sized “B” connector 350, and therefore “A” connector 310 are able to couple to the appropriate socket 392, as illustrated in FIG. 13.

In some embodiments, the subset 192 of “B” connectors 150 and the subset 193 of “A” connectors 110 may include indicia. The indicia may be human or machine readable. The content of the indicia may comprise information related to a patient therapy or medical procedure such as the medical fluids to be transferred, for example. An “B” connector 350 with its key configuration and a “A” connector 310 with a corresponding key receiving configuration may have the same or related indicia content. As such, the indicia may aid in identifying coupleable connector pairs. The sockets 392 may also comprise indicia 394 that corresponds with the appropriate pair of “A” connector 310 and “B” connector 350.

In some embodiments, the differently sized “B” connectors 350 and the corresponding sized “A” connectors 310 may include colored portions comprising two or more different colors. The colors may be associated with the medical fluids to be transferred in the course of a patient therapy or medical procedure. A “B” connector 350 with its key configuration and an “A” connector 310 with a corresponding key receiving configuration may have the same color. As such, the color may aid in identifying coupleable connector pairs. The colors may also correspond to color standard of the clinical setting as may be used to identify fluid types such as saline or other medical fluids. The sockets 392 may also comprise the color code that corresponds with the appropriate pair of “A” connector 310 and “B” connector 350.

It will be obvious to those having skill in the art that many changes may be made to the details of the above-described embodiments without departing from the underlying principles of the invention. The scope of the present invention should, therefore, be determined only by the following claims.

1. A medical fluid connector system, comprising:
a plurality of first connectors of different sizes, each first connector comprising a first fluid lumen; and
a plurality of second connectors of different sizes corresponding with one of the plurality of first connectors, each second connector comprising a second fluid lumen;
wherein each first connector comprises a plurality of key configurations,
wherein each second connector comprises a plurality of key receiving configurations to receive one of the plurality of key configurations of the first connector, and
wherein each second connector comprises a plurality of alignment configurations to properly align the key configurations of first connectors with the key receiving configurations of the second connectors to enable connection between the first connector and the second connector.

2. The connector system of claim 1, wherein the plurality of key receiving configurations and the plurality of alignment configurations are different components.

3. The connector system of claim 1, wherein the second connector comprises a proximal portion, a distal portion, and a flange disposed between the proximal portion and the distal portion, and
wherein the key receiving configurations are disposed on the distal portion of the second connector.

4. The connector system of claim 3, wherein the distal portion of the second connector comprises concentric projections, a nozzle and a skirt, that each extend distally from the flange and define a groove disposed between the nozzle and the skirt to receive a tapered portion of the first connector.

5. The connector system of claim 4, wherein the key receiving configurations comprise two apertures that are disposed in opposing sides of the skirt.

6. The connector system of claim 5, wherein the key configurations comprise two wedges that project radially outward from the body of the first connector, wherein each wedge is configured to snap into a corresponding aperture of the key receiving configurations.

7. The connector system of claim 4, wherein the plurality of alignment configurations comprise two grooves that are disposed on opposing sides of a distal end of the skit.

8. The connector system of claim 7, wherein each groove of the two grooves is longitudinally aligned with a corresponding aperture of the key receiving configurations.

9. The connector system of claim 1, wherein the connection between each of the first connectors and each of the second connectors provides audible feedback.

10. The connector system of claim 1, wherein the connection between each of the first connectors and each of the second connectors provides tactile feedback.

11. The connector system of claim 1, wherein each key receiving configuration is unique with respect to the other key receiving configurations, and
wherein the plurality of key configurations corresponds to the plurality of key receiving configurations in a one to one relationship such that each key receiving configuration is configured to receive its corresponding key configuration and prevent the receiving of all other key configurations.

12. The connector system of claim 11, wherein each key configuration is defined by an external shape of a collar of the first connector, and
wherein each key receiving configuration is defined by an internal shape of an aperture of the second connector.

13. The connector system of claim 12, wherein, upon coupling, the collar of the first connector is at least partially disposed through the aperture of the second connector.

14. The connector system of claim 13, wherein the collar is positionable within the aperture in a single angular orientation of the first connector with respect to the second connector.

15. The connector system of claim 12, wherein each external shape of the collar comprises a plurality of protrusions, a plurality of notches, or a protrusion and a notch disposed along a cylindrical circumference of the collar, and
wherein each internal shape of the aperture comprises a plurality of inward protrusions, a plurality of notches, or an inward protrusion and a notch disposed along a circular perimeter of the aperture.

16. A process of manufacturing a plurality of medical fluid connectors, each connector comprising:
a first fluid lumen disposed along a first longitudinal axis;
an external tapered portion disposed at a distal end of the first lumen; and
one of a plurality of key configurations,
the process comprising:
providing an injection molding machine and a mold base;
providing a molding insert configured to form an outside surface of a distal portion of the connector;
providing a first sub-insert configured to form an outside surface of a proximal portion of the connector defining a first one of the plurality of key configurations;
molding a first plurality of connectors;
replacing the first sub-insert with a second sub-insert configured to form the outside surface of the proximal portion of the connector defining a second one of the plurality of key configurations; and
molding a second plurality of connectors.

17. The process of claim 16, wherein replacing the first sub-insert with the second sub-insert is performed without removing the mold from the injection molding machine.

18. A method of using a system of medical fluid connectors, comprising:
providing a plurality of key connectors, each key connector comprising a first fluid lumen disposed along a first longitudinal axis and an external tapered portion disposed at a distal end of the first lumen;
providing a plurality of key receiving connectors, each key receiving connector comprising a fluid lumen disposed along a second longitudinal axis and an internal tapered portion disposed at a proximal end of the second lumen and sealably coupleable to the external tapered portion,
wherein the plurality of key connectors comprises a plurality of key configurations,
wherein the plurality of key receiving connectors comprises a plurality of key receiving configurations, and
aligning a first key connector with a first key receiving connector such that the key configuration of the first key connector is in longitudinal alignment with the key receiving configuration of the first key receiving connector; and
inserting a distal portion of the first key connector through an aperture of the first key receiving connector;
wherein, upon insertion, engagement of the plurality of key configurations with the plurality of key receiving configurations causes the first key connector to form a fluid tight seal with the first key receiving connector.

19. The method of claim 18, further comprising:
aligning a second key connector with a second key receiving connector such that the key configuration of the second key connector is in longitudinal alignment with the key receiving configuration of the second key receiving connector;
inserting a distal portion of the second key connector through an aperture of the second key receiving connector; and
wherein, upon insertion, engagement of the plurality of key configurations with the plurality of key receiving configurations causes the second key connector to form a fluid tight seal with the second key receiving connector.

20. The method of claim 19, wherein the first and second connectors are coupled to a manifold.
